(19) United States
(12) Patent Application Publication
(10) Pub. No.: US 2025/0009373 A1
(43) Pub. Date: Jan. 9, 2025

(54) CAPTURE BASKET FOR LASER LITHOTRIPSY

(71) Applicant: LUMENIS LTD., Yokneam IIit (IL)

(72) Inventors: Hernan Altman, Tivon (IL); Anat Cohen, Tivon (IL)

(73) Assignee: LUMENIS LTD., Yokneam IIit (IL)

(21) Appl. No.: 18/764,618

(22) Filed: Jul. 5, 2024

Related U.S. Application Data
(60) Provisional application No. 63/512,258, filed on Jul. 6, 2023.

(51) Int. Cl. A61B 17/221; A61B 17/22; A61B 18/00; A61B 18/26

(52) U.S. Cl. CPC A61B 17/221; A61B 18/26; A61B 2017/22074; A61B 2017/22079; A61B 2017/2215; A61B 2018/00511; A61B 2018/00982; A61B 2218/007

(57) ABSTRACT

Devices, systems, and methods for a retrieval device for addressing a kidney stone. A retrieval device includes an expandable basket with a deployed cover. The basket is expanded and maneuvered around the kidney stone. The cover is then deployed around the basket to contain the kidney stone for laser lithotripsy. Fragments at or below a target size are allowed to escape through apertures in the cover or are removed via a suction tube.

### CROSS-REFERENCE TO RELATED APPLICATIONS

This application claims the benefit of U.S. Provisional Patent Application Ser. No. 63/512,258 filed on Jul. 6, 2023, the disclosure of which is incorporated herein by reference.

### TECHNICAL FIELD

This disclosure relates generally to medical devices and, more particularly, to devices for the capture and/or retrieval of objects within a body, such as stones, other calculi, or foreign matter.

### BACKGROUND

Retrieval devices have been used to remove stones and other calculi from within the body of a patient. One type of retrieval device includes a sheath, an elongate member extending within and moveable relative to the sheath, and a basket connected to the distal end of the elongate member. The basket may have a number of legs and may have a collapsed position within the sheath and an expanded position beyond a distal end of the sheath. When the basket is in the expanded position, a targeted stone may be captured within the basket and removed from the body. Some stones, however, may be too large to be removed from the body after being captured within the basket. In this situation, a device, such as a laser fiber or other laser lithotripsy device, may be used to break up the stone before the extraction (referred to herein as “stone communition”). The stone fragments may then be removed from the body without harming the surrounding body tissue.

If stone lasering is attempted while the stone is inside a basket, the smaller stone fragments generated during use of the laser may escape the basket, escaping between the struts. These stone fragments may be greater than an acceptable size and can migrate outside of the immediate working area, often lodging elsewhere in the urinary tract. If left, these larger fragments may cause problems for the patient; finding and recapturing them, on the other hand, may substantially extend the duration of the surgical procedure.

The present disclosure provides retrieval devices and methods of using the same that avoid the aforementioned shortcomings of existing devices.

### SUMMARY

This disclosure provides design, material, manufacturing method, and use alternatives for medical devices and medical systems. In a first example, a retrieval device for addressing a kidney stone, can comprise an elongate member; a basket connected to a distal end of the elongate member, the basket moveable between a retracted position having a first diameter and an expanded position having a second diameter larger than the first diameter; and a cover connected to the basket, the cover deployable to form a barrier around the basket after the basket has moved into the expanded position and surrounded a kidney stone.

Alternatively or additionally to any of the examples above, the basket can include a sheath sized to enclose the basket in the retracted position, wherein the basket exits the sheath when moving into the expanded position.

Alternatively or additionally to any of the examples above, the basket can include a plurality of legs joined at a proximal end to the elongate member and at the distal end to an atraumatic tip.

Alternatively or additionally to any of the examples above, the device can further include a plurality of strings connected to the cover, the strings running between the cover and the atraumatic tip of the basket such that pulling the strings deploys the cover.

Alternatively or additionally to any of the examples above, the cover can move from a proximal end of the basket to the atraumatic tip to form the barrier around the basket.

Alternatively or additionally to any of the examples above, the device can further include a plurality of strings connected to the cover, wherein the cover is deployed ahead of the basket such that pulling the strings moves the cover from a distal end of the basket to a proximal end of the basket to form a barrier around the basket.

Alternatively or additionally to any of the examples above, the cover can be water-permeable.

Alternatively or additionally to any of the examples above, the cover can include at least one sheet of biocompatible material.

Alternatively or additionally to any of the examples above, the biocompatible material can be thermoplastic, plant fiber, or animal tissue.

Alternatively or additionally to any of the examples above, the cover can have a plurality of holes of a preselected size.

Alternatively or additionally to any of the examples above, the holes can be between 1 mm and 5 mm in diameter.

Alternatively or additionally to any of the examples above, the cover can be a wire mesh, the holes comprising spaces between wires of the mesh.

In another example, an endoscopic surgical device comprises an endoscope having an instrument channel; a retrieval device according to any of the examples above, wherein the first diameter of the basket of the retrieval device in the retracted position is less than an internal diameter of the instrument channel such that the retrieval device can be deployed into a patient's body through the instrument channel of the endoscope; and a laser fiber suitable to deploy through the endoscope and insert into a working space formed by the expanded basket and deployed cover of the retrieval device.

Alternatively or additionally to any of the examples above, the endoscopic surgical device can further include a suction tube suitable to deploy through the endoscope and insert into the working space, the suction tube in fluid communication with a source of suction.

In another example, a method for operating an endoscopic surgical device of any of the examples above comprises expanding the basket of the retrieval device; maneuvering the basket around a target object; deploying the cover to surround the basket with the target object inside, the covered basket defining a working space; inserting a laser fiber into the working space; and applying laser energy through the laser fiber to reduce the target object to one or more fragments.

In another example, an endoscopic surgical device comprises an endoscope having an instrument channel; a retrieval device, comprising an elongate member, a basket connected to a distal end of the elongate member, the basket moveable between a retracted position having a first diameter less than an internal diameter of the instrument channel and an expanded position having a second diameter larger than the internal diameter of the instrument channel, and a cover connected to the basket, the cover deployable to form a barrier around the basket after the basket has moved out of the instrument channel and into the expanded position and surrounded a kidney stone; and a laser fiber suitable to deploy through the endoscope and insert into a working space formed by the expanded basket and deployed cover of the retrieval device.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a retrieval device within a body lumen;

FIG. 2 depicts the retrieval device of FIG. 1 in an expanded position;

FIG. 3 depicts the retrieval device of FIGS. 1 and 2 with a captured kidney stone;

FIG. 4 depicts the retrieval device of FIGS. 1-3 with a deployed cover.

FIG. 5 depicts the retrieval device of FIGS. 1-4 with a laser fiber addressing the kidney stone;

FIG. 6 depicts the retrieval device of FIGS. 1-5 with a suction tube removing stone fragments;

FIG. 7 depicts a retrieval device with an alternative cover;

FIGS. 8A and 8B depict the deployment of a cover around a basket of a retrieval device;

FIGS. 9A-9E depict an alternative deployment of a cover around a basket of a retrieval device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

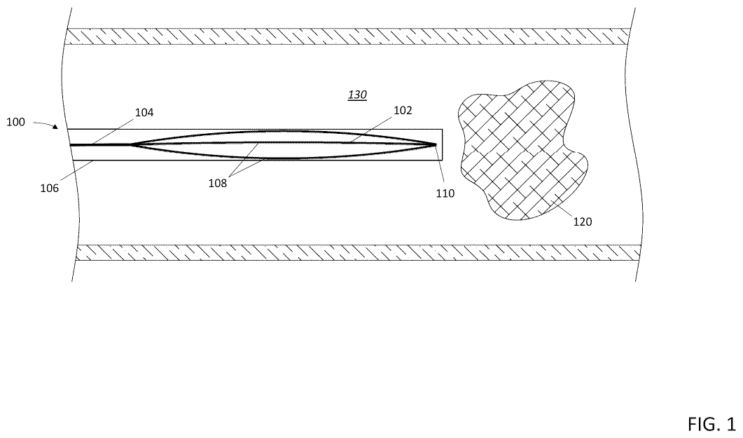

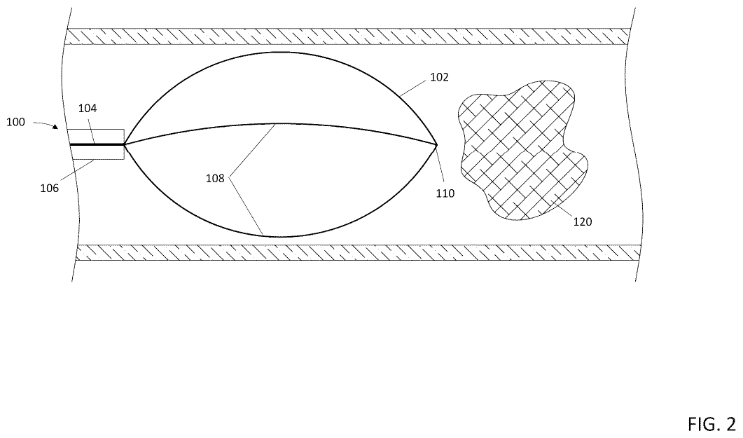

FIGS. 1 and 2 illustrate a retrieval device 100 according to an exemplary embodiment of the present disclosure. The retrieval device 100 includes a basket 102 connected to an elongate member 104. The device 100 further includes a sheath 106, and the elongate member 104 is disposed within the sheath 106. Relative movement between the elongate member 104 and the sheath 106 assists the basket 102 in forming an expanded position (shown in FIG. 2) where the basket 102 is disposed substantially completely beyond a distal end of the sheath 106, and a retracted position (shown in FIG. 1) where the basket 102 is disposed substantially completely within the sheath 106.

In some implementations, the device 100 may not include the sheath 106. Instead, the component designated as sheath 106 in the figures may instead be an instrument channel 106 associated with an endoscopic probe. One of ordinary skill in the art will recognize that moving the elongate member 104 relative to the channel 106 of the endoscopic probe will allow for retracted and expanded positions of the basket 102 as described herein.

The basket 102 may include a plurality of legs 108. The legs 108 and, thus, a proximal end of the basket 102 may be connected to a distal end of the elongate member 104 in any conventional way. For example, the legs 108 may be made of separate pieces of material that are welded, glued, braided, soldered, and/or otherwise connected to the distal end of the elongate member 104. Alternatively, in another exemplary embodiment, the legs 108 may be formed of the same piece or pieces of material as the elongate member 104. Although the figures show a basket 102 having three legs 108, other exemplary embodiments of the basket 102 may include more or fewer than three legs 108 to facilitate the retrieval of a stone 120, calculi, or other foreign matter.

In an exemplary embodiment, the legs 108 may be looped, knotted, tied, braided, soldered, welded, glued, and/or otherwise joined at a distal end of the basket 102, and the distal end may define an atraumatic tip 110. The atraumatic tip 110 may be blunt, rounded, flat, smooth, knotted, looped, or any other atraumatic shape known in the art. The atraumatic tip 110 may be sized to assist in the capture and retrieval of stones 102 within the body. For example, the atraumatic tip 110 may be as small as possible so as not to interfere with the capture and retrieval of the targeted stone 120. In an exemplary embodiment, the legs 108 of the basket 102 may form the atraumatic tip 110. In an alternative embodiment, the atraumatic tip 110 may comprise, for example, a cap.

The elongate member 104 may be formed from, for example, a wire, rod, tube, hypotube, cannula, stent, or other piece of biocompatible material or combination of biocompatible materials known in the art. Such materials may include, but are not limited to, polyamide, PEBAX, silver, stainless steel (such as 300 and 400 series), cobalt chromium, nickel, titanium, nitinol, thermoforming plastic, polytetrafluoroethylene (“PTFE”), and expanded polytetrafluoroethylene (“ePTFE”). The elongate member 104 may also be a metal coated with a polymer and may have one or more layers of material. The elongate member 104 may be solid or hollow, and may be substantially cylindrical. Alternatively, the elongate member 104 may be formed from a flat sheet of material. If formed from a flat sheet, the elongate member 104 may be formed into a substantially cylindrical shape.

The overall length and diameter of the elongate member 104 may vary depending on the application. For example, a relatively long elongate member 104 may be advantageous for retrieving stones 120 or other calculi deep within the body of the patient. In addition, an elongate member 104 having a relatively small diameter may be advantageous for retrieving stones 120 from restricted passageways within the human urinary tract. The elongate member 104 may be relatively flexible to facilitate the retrieval of stones 120 located in complex body structures.

The sheath 106 may be formed from any of the materials discussed above with respect to the elongate member 104. The sheath 106 may be dimensioned to fully enclose the elongate member 104 and the basket 102 when the basket 102 is in the retracted position shown in FIG. 1. Where the device 100 lacks a sheath 106, the elongate member 104 and the basket 102 may be sized so that, in the retracted position, they fit within the instrument channel 106 of an endoscopic probe. It is known for endoscopic probes to include an instrument channel having a standard diameter, which can be used as a design constraint to the diameter of the device 100 in the retracted position.

Each of the legs 108 may be discrete wires, fibers, strands, filaments, or like elongate structures that are joined together to form the basket 102. Alternatively, the legs 108 may be cut, etched, removed, stamped, and/or otherwise formed from a single piece of material.

With reference to FIG. 1, the retrieval device 100 may be first inserted in a body lumen 130 while in a retracted position such that the basket 102 is fully within the sheath 106 or endoscope instrument channel 106. The retrieval device 100 may be deployed within an endoscope of any appropriate sort known in the art, which may include imaging and guidance functions to locate a stone 120 to be removed.

The retrieval device 100 is moved near the stone 120 and then expanded, as shown in FIG. 2. By holding the elongate member 104 and basket 102 stationary while retracting the sheath 106, or by moving the device 100 past the end of the endoscope instrument channel 106, the basket 102 is permitted to open into its biased position. In some implementations, mechanical or motorized elements may further aid the basket 102 in expanding.

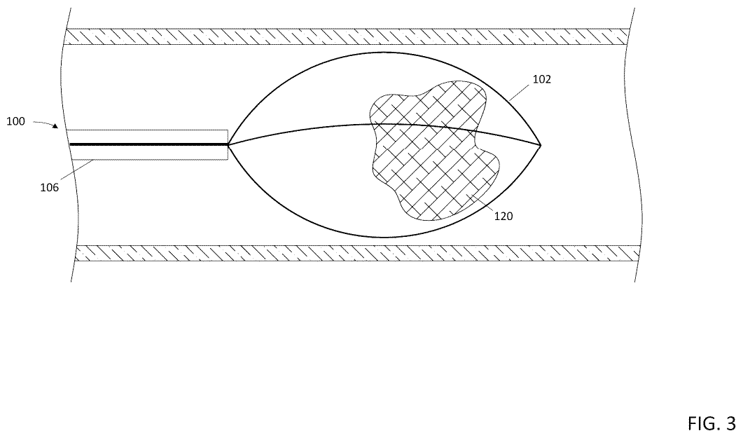

Once the retrieval device 100 is expanded, the basket 102 is maneuvered to surround the target stone 120 as shown in FIG. 3. Maneuvering the basket may be accomplished by the use of imagers, guidewires, or other endoscopic tools as known in the art.

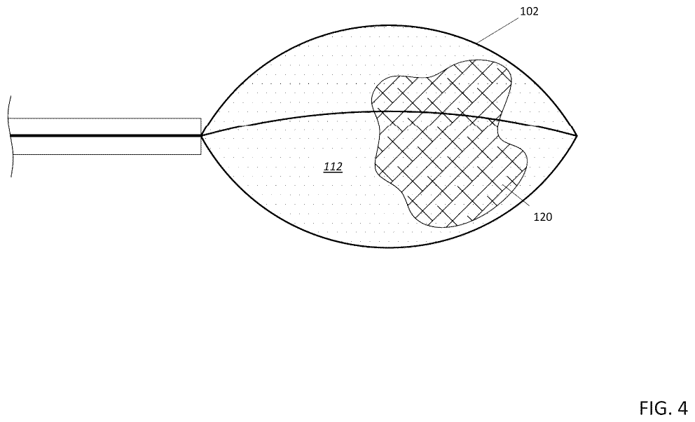

A cover 112 is then deployed over the basket 102, as illustrated in FIG. 4. The cover 112 may be made of cloth, plastic, or of any other biocompatible material known in the art. Where the retrieval device 100 is single-use, a relatively inexpensive material, such as polyethylene terephthalate or polytetrafluoroethylene plastic may be used. Plant fiber, such as cotton or hemp, or denatured animal tissue, such as mammalian pericardium, may also be employed in some procedures. The basket cover may be water-permeable, but notably is not permeable to stone fragments during laser communition, as further described herein. A mesh material such as stainless steel may be used in some embodiments for the cover; the hole size of the mesh may act as a size-filtration barrier for stone fragments as further described below.

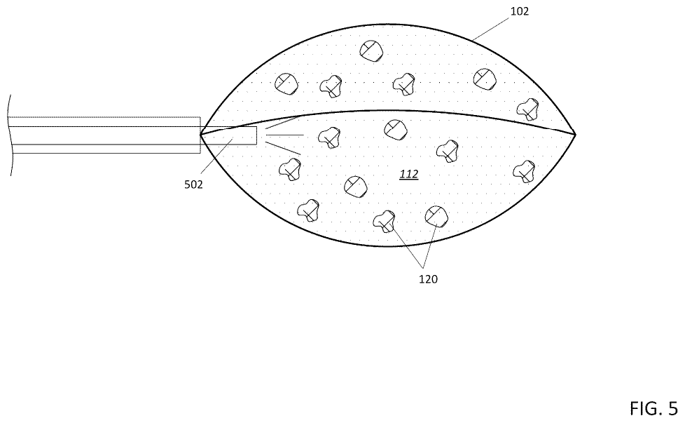

As shown in FIG. 5, once the cover 112 is deployed around the basket 102, a laser fiber 502 is inserted. The laser fiber 502 conducts energy from a laser source appropriate for a lithotripsy procedure. Any of a variety of solid-state pulsed lasers, such as a Holmium: YAG laser or thulium fiber laser, may be employed to address the stone 120.

Various techniques for laser lithotripsy are known. The stone 120 is reduced to fragments 122 through ablation, fragmentation, and/or dusting. The basket cover 112 contains the fragments 122 during the communition process. The stone communition may continue until fragments 122 are below a target size, such as 5 mm. Once the fragments have reached the permitted size, the fragments may then be released or removed.

In some implementations, other methods of communition may be used instead of, or in addition to, laser communition, as known in lithotripsy procedures. For example, both hydraulic and ultrasound methods of lithotripsy are known in the art. The device 100 may be shaped and designed to withstand the application of these energies to break up a stone 120 and/or fragments 122.

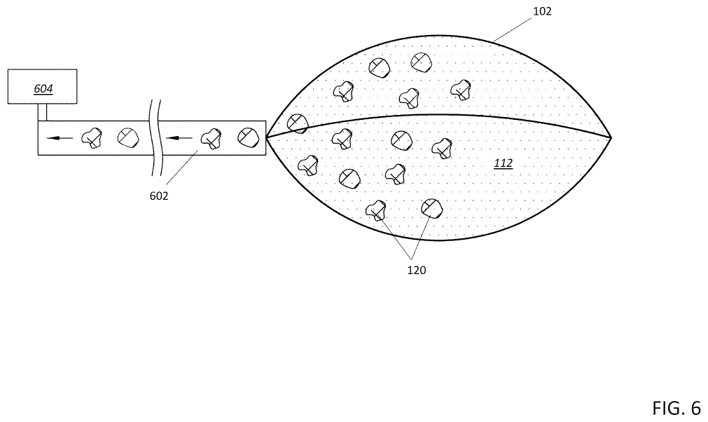

In one implementation, illustrated in FIG. 6, stone fragments are removed through a suction tube 602 in communication with a source of suction 604. The suction tube 602 may be delivered through an endoscope, as described above with respect to delivery of the retrieval device 100. The target size for the fragments 122 may depend on the size of the suction tube 302, which may in turn be limited by an instrument channel size in the endoscope. In some implementations, the suction may be applied intermittently during the use of the laser, removing fragments 122 that are small enough for the suction tube 602 while leaving larger fragments for further targeting.

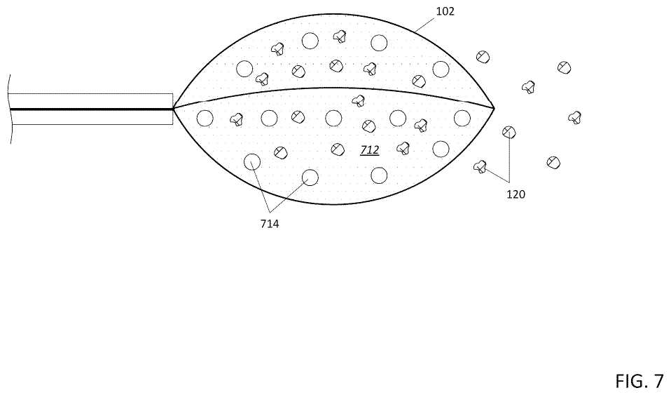

FIG. 7 shows a basket cover 712 with apertures 714 therein. The apertures 714 are sized deliberately to match a stone communition size, such as 2 mm. Fragments 122 larger than the target size are contained within the basket 102 by the basket cover 712. Fragments 122 smaller than the target size can leave the basket 102 through the apertures 714. During laser targeting of the stone 120 and later the fragments 122, the retrieval device 100 may be moved in order to facilitate escape of the smaller fragments 122. For example, partial retraction of the basket 102 into the sheath or channel 106 would partially collapse the basket 102, encouraging expulsion of debris. Similarly, moving or rotating the basket 102 could cause small fragments 122 to escape the basket 102. Rather than a substantially solid cover with holes, a mesh cover could also have the holes between wires of the mesh spaced to match the target size of the fragments.

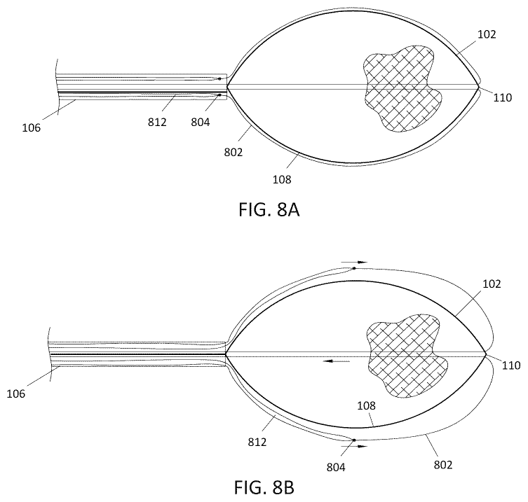

FIGS. 8A and 8B illustrate an implementation for deployment of a cover 812. FIG. 8A shows the basket 102 already free of the sheath or instrument channel 106 and in the expanded position, while the cover 812 is still fully within the sheath or channel 106. Strings 802 run along the legs 108 of the basket 102 from tie points 804 to the atraumatic tip 110, and then back up the center of the basket 102 and through the sheath or channel 106. The strings 802 may further extend into the sheath or channel 106 and back up the instrument channel for direct manipulation by a medical practitioner, or may be affixed to a device for automated manipulation. The cover 812 is also affixed to each of the tie points 804. As shown in FIG. 8B, pulling the portion of the string in the center of the basket 102 causes the outside portions of the string 802 to move towards the basket tip 110. This, in turn, pulls the tie points 804 and cover 812 distally along the legs 108. The strings 802 are pulled until the tie points 804 reach the tip 110, thus deploying the cover 812 along the entire outside of the basket 102.

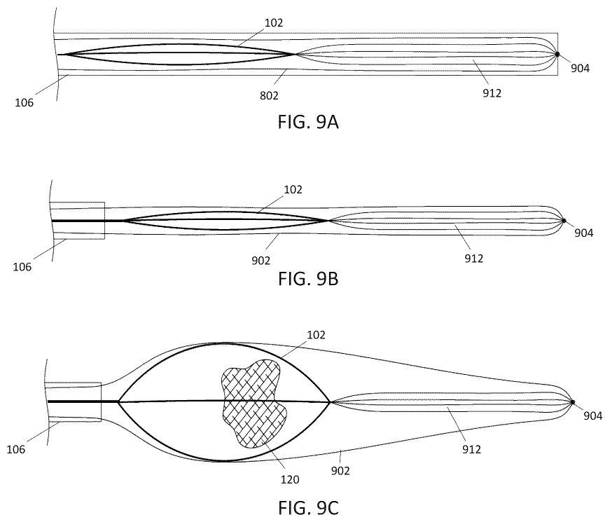

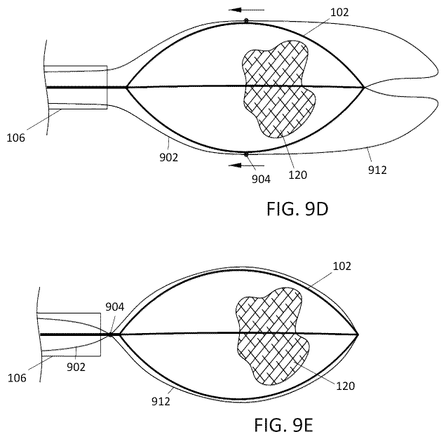

FIGS. 9A through 9E illustrate an implementation for deployment of a cover 912. FIG. 9A shows the cover 912 in the sheath or channel 106 forward of the basket 102. When the retrieval device is moved into the open position, the cover 912 emerges from the sheath or channel 106 first, as shown in FIG. 9B. Once both the cover 912 and the basket 102 have emerged, the basked can open and maneuver to capture the stone 120 as shown in FIG. 9C. Strings 902 are attached to the cover 912 at tie points 904 at the distal end. As shown in FIGS. 9D and 9E, pulling the strings 902 proximally towards the sheath 106 results in the cover 912 moving over the basket 102 to its deployed position.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A retrieval device for addressing a kidney stone, comprising:
an elongate member;
a basket connected to a distal end of the elongate member, the basket moveable between a retracted position having a first diameter and an expanded position having a second diameter larger than the first diameter; and
a cover connected to the basket, the cover deployable to form a barrier around the basket after the basket has moved into the expanded position and surrounded a kidney stone.

2. The retrieval device of claim 1, further comprising:
a sheath sized to enclose the basket in the retracted position, wherein the basket exits the sheath when moving into the expanded position.

3. The retrieval device of claim 2, the basket comprising:
a plurality of legs joined at a proximal end to the elongate member and at the distal end to an atraumatic tip.

4. The retrieval device of claim 3, further comprising:
a plurality of strings connected to the cover, the strings running between the cover and the atraumatic tip of the basket such that pulling the strings deploys the cover.

5. The retrieval device of claim 4, wherein cover moves from a proximal end of the basket to the atraumatic tip to form the barrier around the basket.

6. The retrieval device of claim 1, further comprising:
a plurality of strings connected to the cover, wherein the cover is deployed ahead of the basket such that pulling the strings moves the cover from a distal end of the basket to a proximal end of the basket to form a barrier around the basket.

7. The retrieval device of claim 1, wherein the cover is water-permeable.

8. The retrieval device of claim 1, wherein the cover comprises at least one sheet of biocompatible material.

9. The retrieval device of claim 8, wherein the biocompatible material is thermoplastic, plant fiber, or animal tissue.

10. The retrieval device of claim 1, wherein the cover has a plurality of holes of a preselected size.

11. The retrieval device of claim 10, wherein the holes are between 1 mm and 5 mm in diameter.

12. The retrieval device of claim 10, wherein the cover is a wire mesh, the holes comprising spaces between wires of the mesh.

13. An endoscopic surgical device, comprising:
an endoscope having an instrument channel;
a retrieval device, comprising:
an elongate member,
a basket connected to a distal end of the elongate member, the basket moveable between a retracted position having a first diameter less than an internal diameter of the instrument channel and an expanded position having a second diameter larger than the internal diameter of the instrument channel, and
a cover connected to the basket, the cover deployable to form a barrier around the basket after the basket has moved out of the instrument channel and into the expanded position and surrounded a kidney stone; and
a laser fiber suitable to deploy through the endoscope and insert into a working space formed by the expanded basket and deployed cover of the retrieval device.

14. The endoscopic surgical device of claim 13, further comprising:
a suction tube suitable to deploy through the endoscope and insert into the working space, the suction tube in fluid communication with a source of suction.

15. The endoscopic surgical device of claim 13, the basket of the retrieval device comprising:
a plurality of legs joined at a proximal end to the elongate member and at the distal end to an atraumatic tip.

16. The endoscopic surgical device of claim 13, the retrieval device comprising:
a plurality of strings connected to the cover, the strings running between the cover and the atraumatic tip of the basket such that pulling the strings deploys the cover.

17. The endoscopic surgical device of claim 16, wherein cover of the retrieval device moves from a proximal end of the basket to the atraumatic tip to form the barrier around the basket.

18. The retrieval device of claim 13, the retrieval device comprising:
a plurality of strings connected to the cover, wherein the cover is deployed ahead of the basket such that pulling the strings moves the cover from a distal end of the basket to a proximal end of the basket to form a barrier around the basket.

19. A method for operating an endoscopic surgical device, comprising:
inserting an endoscopic surgical device into a patient's body, the endoscopic surgical device comprising:
an endoscope having an instrument channel;
a retrieval device, comprising:
an elongate member,
a basket connected to a distal end of the elongate member, the basket moveable between a retracted position having a first diameter less than an internal diameter of the instrument channel and an expanded position having a second diameter larger than the internal diameter of the instrument channel, and
a cover connected to the basket, the cover deployable to form a barrier around the basket after the basket has moved out of the instrument channel and into the expanded position and surrounded a kidney stone, and
a laser fiber suitable to deploy through the endoscope and insert into a working space formed by the expanded basket and deployed cover of the retrieval device;
expanding the basket of the retrieval device;
maneuvering the basket around a kidney stone;
deploying the cover to surround the basket with the kidney stone inside, the covered basket defining a working space;
inserting a laser fiber into the working space; and
applying laser energy through the laser fiber to reduce the kidney stone to one or more stone fragments.

20. The method of claim 19,
wherein the endoscopic surgical device further comprises a suction tube suitable to deploy through the endoscope and insert into the working space, the suction tube in fluid communication with a source of suction; and
wherein the method further comprises:
inserting the suction tube into the working space through the endoscope; and
removing the one or more stone fragments through the endoscope via the suction tube.
